SEQUENCE LISTING (5 sequences)

SEQ ID NO: 1 - protein, 87 aa, Homo sapiens
MTRYFCCGSYFPGYPIYGTNFHGTFRATPLNCVVPLGSPLNYGCGCNGYSSLGYSFGGSNINNLGGCYGGSFYRPWGSGSGFGYSTY

SEQ ID NO: 2 - protein, 63 aa, Homo sapiens
MLCDNFPGAVFPGCYWGSYGYPLGYSVGCGYGSTYSPVGYGFGYGYNGCGAFGYRRYSPFALY

SEQ ID NO: 3 - protein, 90 aa, Homo sapiens
MSHYGSYYGGLGYSCGGFGGLGYGYGCGCGSFCRRGSGCGYGGYGYGSGFGSYGYGSGFGGYGYGSGFGGYGYGCCRPSYNGGYGFSGFY

SEQ ID NO: 4 - protein, 52 aa, Homo sapiens
MCYGYGCGCGSFCRLGYGCGYEGCRYGCGHRGCGDGCCCPSCYRRYRFTGFY

SEQ ID NO: 5 - protein, 229 aa, Homo sapiens
MSFLQDPSFFTMGMWSIGAGALGAAALALLLANTDVFLSKPQKAALEYLEDIDLKTLEKEPRTFKAKELWEKNGAVIMAVRRPGCFLCREEAADLSSLKSMLDQLGVPLYAVVKEHIRTEVKDFQPYFKGEIFLDEKKKFYGPQRRKMMFMGFIRLGVWYNFFRAWNGGFSGNLEGEGFILGGVFVVGSGKQGILLEHREKEFGDKVNLLSVLEAAKMIKPQTLASEKK

(19) United States
(12) Patent Application Publication
(10) Pub. No.: US 2019/0091370 A1
(43) Pub. Date: Mar. 28, 2019

(54) Mechanism of Action of Glycine and Tyrosine KAPs in Engineered Matrices from Human Hair

(71) Applicant: Cell Constructs I, LLC, Marietta, GA (US)

(72) Inventors: Mitchell C. Sanders, Grafton, MA (US); Thomas H. Barrows, Austell, GA (US)

(21) Appl. No.: 16/125,931

(22) Filed: Sep. 10, 2018

Related U.S. Application Data
(60) Provisional application No. 62/556,638, filed on Sep. 11, 2017.

(51) Int. Cl. A61L 27/36; A61K 35/28; A61L 27/38; A61P 17/02; A61L 27/52; C12N 5/00; A61K 35/36

(52) U.S. Cl. CPC A61L 27/3604; A61K 35/28; A61L 27/3834; C12N 2533/50; A61L 27/52; C12N 5/0068; A61K 35/36; A61P 17/02

(57) ABSTRACT

Disclosed herein is a wound dressing having a wound-contacting surface comprising a KAP-enriched keratin material in the form of a hydrogel or a coating on a substrate wherein said KAP content facilitates accelerated wound healing of chronic wounds that exhibit excessive or persistence proteolytic enzyme activity.

### RELATED APPLICATION

This patent application is a Non-Provisional Application which claims the benefit of U.S. Provisional Application No. 62/556,638 filed on Sep. 11, 2017, entitled “Mechanism of Action of Glycine and Tyrosine KAPs in Engineered Matrices from Human Hair,” which is incorporated herein by this reference in its entirety.

### TECHNICAL FIELD

The technical field includes wound dressings, and in particular, biomaterials suitable for contact with open wounds that facilitate optimal wound healing.

### BACKGROUND

Most superficial wounds involving broken skin on healthy individuals heal rapidly without the need for intervention beyond keeping the wound protected and free of contamination. However, many conditions such as old age, diabetes, poor circulation of blood in the legs, and pressure ischemia as seen in bed-ridden invalids can delay or even prevent wound healing. Dealing with such chronic wounds continues to be a major unsolved medical problem causing untold human suffering and an enormous cost burden to the healthcare system.

Approximately 4 to 5 million people in the US suffer from chronic wounds such as diabetic foot ulcers (DFU), pressure ulcers, and venous leg ulcers (VLU). These chronic wounds often take greater than 60 days to heal because they are unable to progress from the inflammatory or early proliferative phases of wound healing into the tissue reparative phase that ultimately achieves wound closure. Recent clinical studies (Serena, et al. 2016) suggest that having elevated protease activity (EPA) in chronic wounds can stall the wound healing process in the inflammatory phase. Wounds greater than 2 cm² having elevated levels of matrix metalloproteinases (MMPs) and/or human neutrophil elastase predictably are destined to become stalled in the inflammatory phase of wound healing.

In recent years, human proteins extracted from hair clippings (an unregulated waste material) have become recognized as having biological activity important for wound healing.¹ The reversible, molecular self-assembly attribute of keratins and keratin-associated proteins has previously been exploited to make a clear matrix that is purely comprised of reconstituted human proteins without the use of additives or chemical crosslinking agents², now branded as ProgenaMatrix™, which demonstrated wound-healing efficacy on par with allograft human tissues.³

Human hair is composed of keratin intermediate filaments and 3 classes of keratin associated proteins (KAPs). KAPs can be classified by high cysteine content (≤30% cysteine), ultra-high cysteine content (>30% cysteine), and glycine and tyrosine rich proteins. Human hair is composed of 5% gly/tyr KAPs and gly/tyr KAPs are thought to be important for stiffness, diameter, and straightness of human and animal hair (Li et al., 2017, Matsunaga 2009, Zhao et al., 2009). Studies by Rogers et al., (2002) suggest that KAPs are differentially expressed in the cortex of human hair. KAPs 7.1, 8.1, 19.1 and 19.2 are highly expressed in the cortex of human hair whereas KAP 11.1 is highly expressed in beards.

The structure of KAP 8.1 was recently determined by Singh et al. (2017). KAP 8.1 has a Greek-key motif with intercalated anti-parallel beta sheets. Based on structural similarity to gamma-D-crystallin, a molecule demonstrated previously to be a protease inhibitor, it is possible that gly/tyr rich KAPs in human hair are protease inhibitors (Sanders 2000, Sanders et al., 2005). Tyr/Gly rich KAPs have limited sequence identity to gamma-D-crystallin and alpha crystallin/small molecular chaperone type proteins, but its structural similarity to gamma-D-crystallin, which has two Greek-key motifs separated by a flexible linker, is quite apparent.

Accordingly, a need exists for an advanced wound dressing that utilizes the KAP content of hair to naturally reduce or inhibit the excessive and persistent proteolytic enzyme activity found in chronic wounds, thereby facilitating a normal progression and resolution of the inflammatory phase of healing and faster wound closure.

### SUMMARY

An aspect is a wound dressing comprising a wound-contacting surface comprised of keratin proteins extracted from human hair such that the mixture of keratin proteins is enriched with KAPs.

Another aspect comprises a mesh or other suitable substrate coated with KAP-enriched keratin proteins on the wound-contacting side. Said substrate may contain therapeutic and/or antiseptic ingredients. Thus, a method is provided to utilize keratin hydrogel, or keratin precipitated from solution to create a wound dressing that places KAP-enriched keratin in intimate contact with the wound.

### BRIEF DESCRIPTION OF THE FIGURES

FIG. 1. Full thickness wounds in a porcine model were allowed to heal with either ProgenaMatrix™ (presumed to contain at least a natural abundance of KAPs), Promogran®, or an inactive Tegaderm® cover dressing (-control). The ProgenaMatrix™ had the highest wound closure rate and the least amount of periwound redness (erythema).

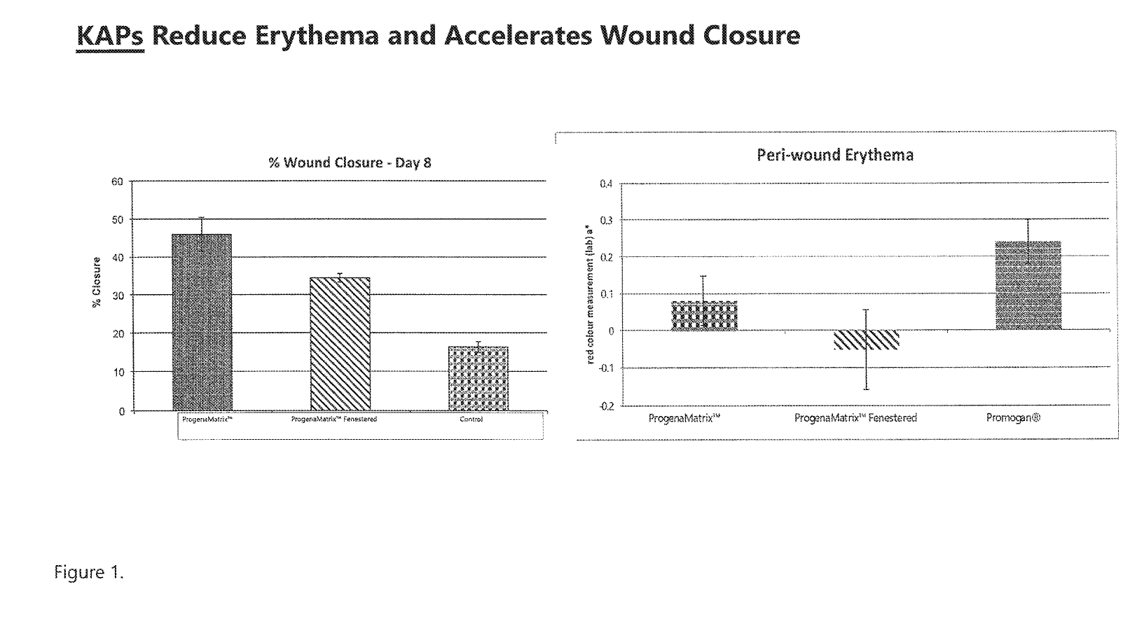

FIG. 2. Sequences of KAPs expressed in the cortex of human hair. The bold letters denote the richness of glycine and tyrosine

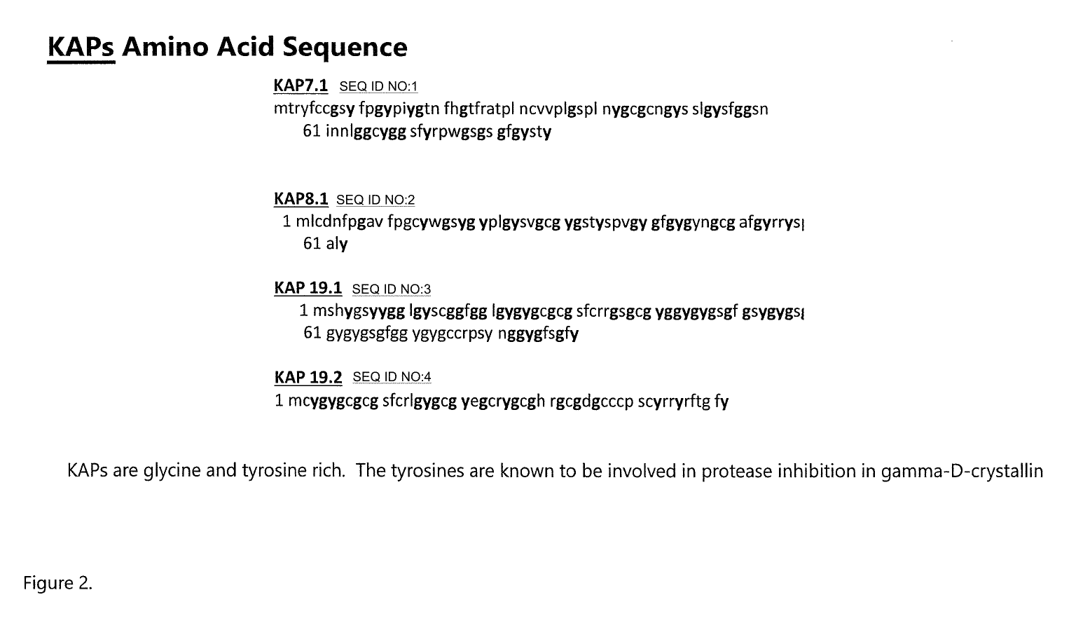

FIG. 3. In the porcine wound healing study described in FIG. 1, elevated levels of protease were detected using the elevated protease activity (EPA) diagnostic from WoundChek Labs. We determined in vivo that KAPs dramatically reduces the EPA of neutrophil elastase and MMPs when compared against the Promogran® and the negative control.

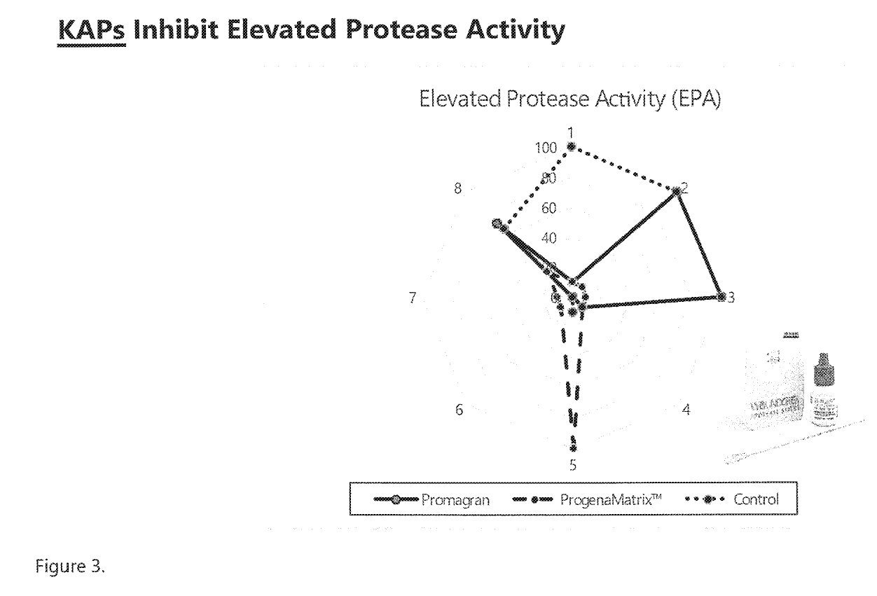

FIG. 4. Summary of findings from Rogers et al., 2002. KAPs are a multi-gene family with differential gene expression. KAP 11.1 is prominently found in beards whereas 7.1, 8.1 19.1 and 19.2 are found in human hair. Many of the genes in this multi-gene family are not expressed in beards or human hair.

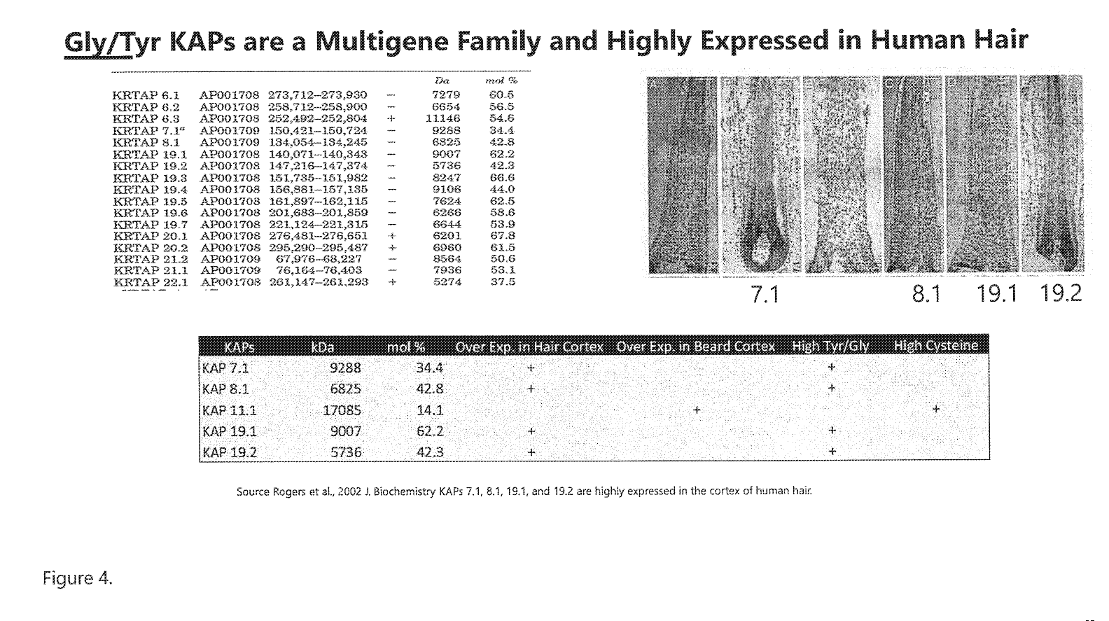

FIG. 5. KAPs have very limited sequence identity that makes typical computer alignments of the sequences extremely problematic. We used the program T-Coffee that uses source tree based consistency objective function for alignment evaluation of sequences with limited identity (Magis et al., 2012).

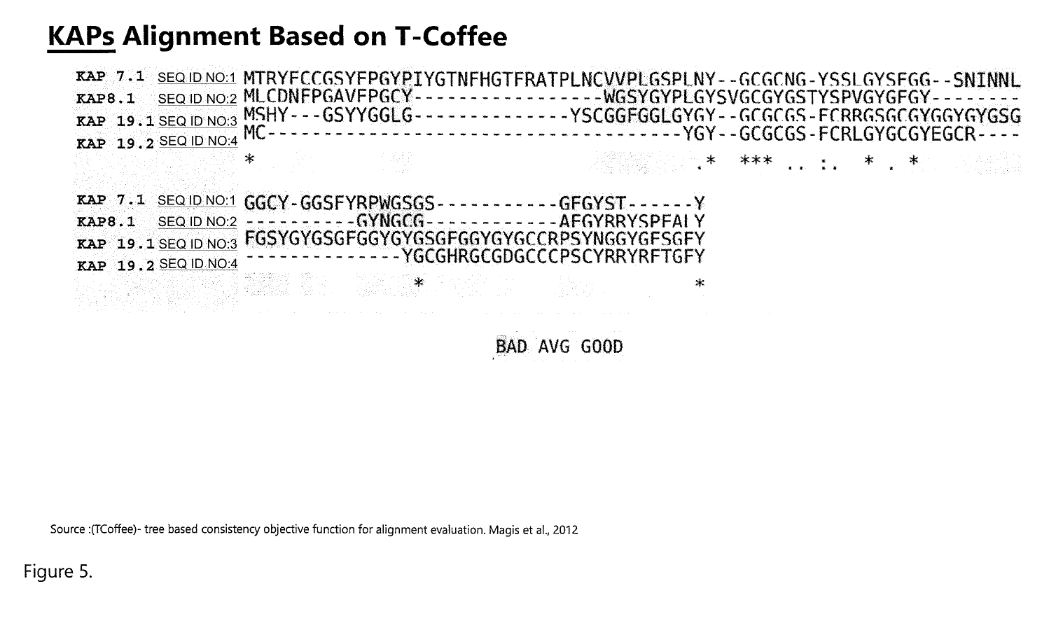

FIG. 6. Greek-key motif with intercalated anti-parallel beta sheets of SMPI, KAPs, and Gamma D Crystallin. SMPI is a serine metalloprotease inhibitor from Streptomyces nigrescens. It has the same structural fold as KAPs, KAPs have a similar Greek-key motif with intercalated anti-parallel beta sheet structure with glycine and tyrosine on the surface. The structure is similar to both domains of gamma D crystallin that has two Greek key motifs separated by a flexible linker.

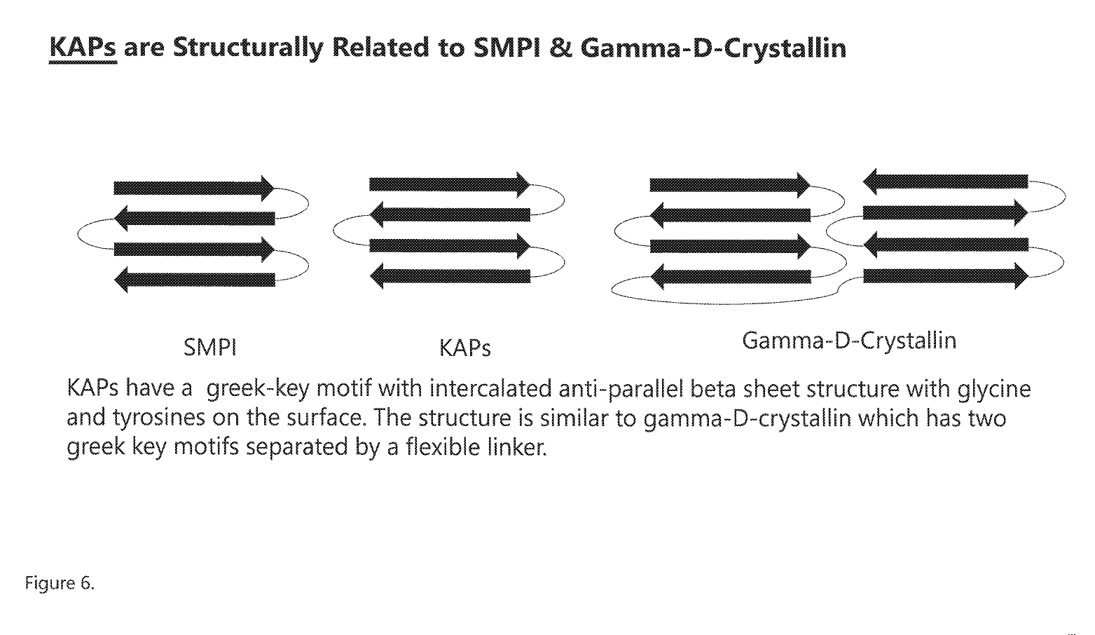

FIG. 7. Synthetic peptide fragments of KAPs 8.1, and 19.2 were used to demonstrate the inhibition of human neutrophil elastase. Peptide fragments of KAPs 8.1 and 19.2 reduce the HNE activity by 36-38%. The data represents three replicates and the error bars represent the standard deviation of the mean. This demonstrates that KAPs are protease inhibitors in vitro as well as in vivo.

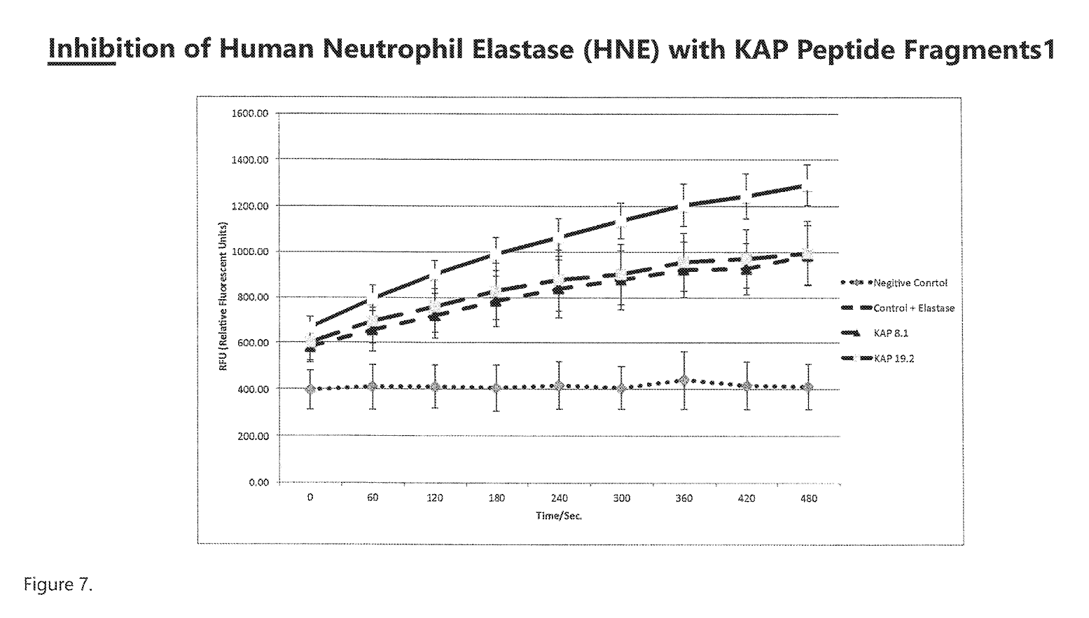

FIG. 8. A dot blot analysis of ProgenaMatrix™ stained with anti-KAP 8.1 antibodies demonstrate that ProgenaMatrix™ contains the glycine and tyrosine rich KAP 8.1.

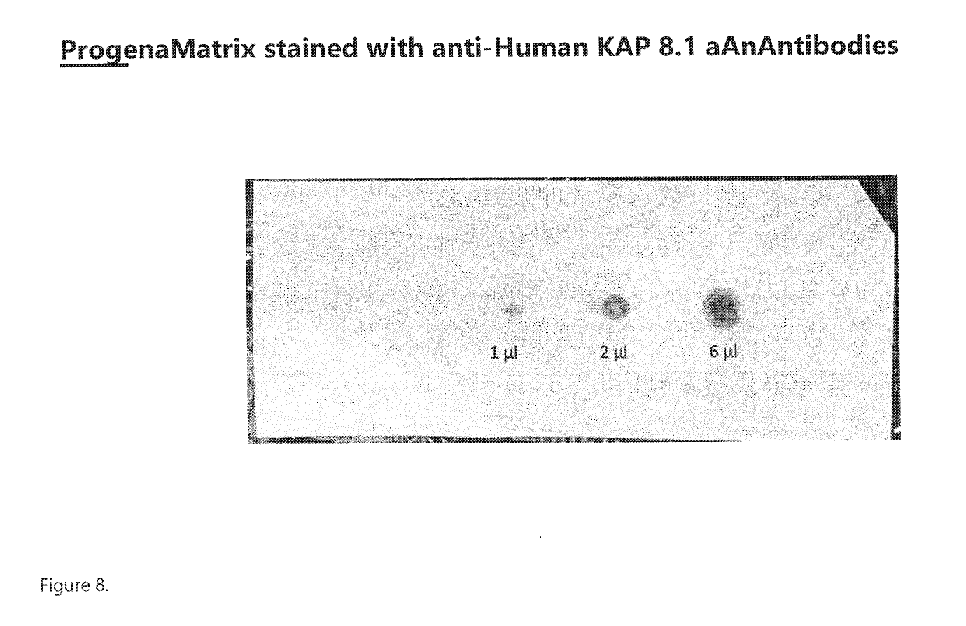

FIG. 9. Luminex studies indicate that anti-inflammatory cytokines IL-6, IL-10 and IL-12 are overexpressed in the full thickness porcine wound healing model when the wounds were treated with ProgenaMatrix™ that contains KAPs but not Promogran® or the untreated −control.

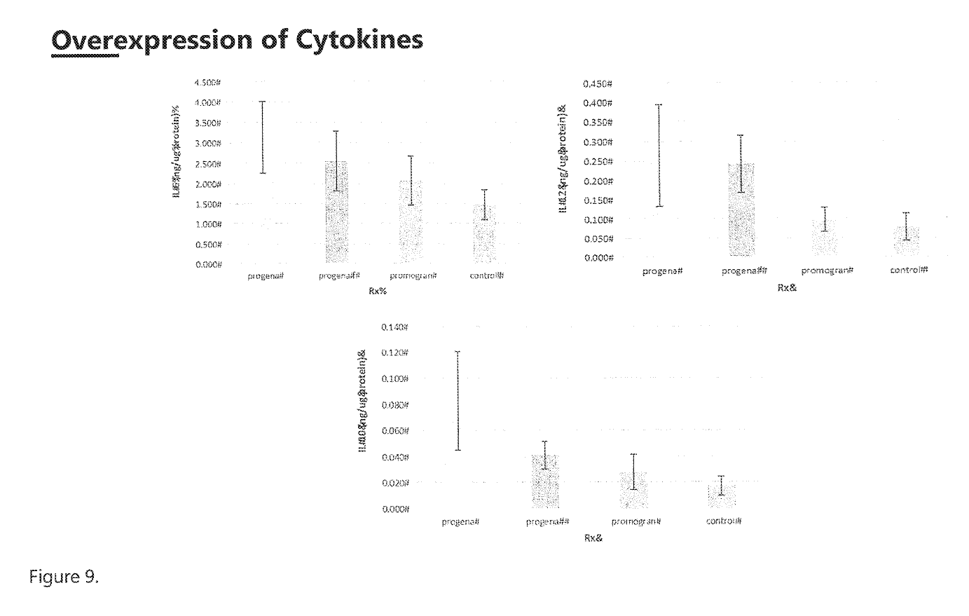

FIG. 10. KAP 8.1 has good structural homology with PAMM, a peroxiredoxin (PRX)-like 2 activated in M-CSF stimulated monocytes protein that has been determined by Guo et al., (2015) to be involved in inhibiting macrophage inflammation by inhibiting MAPK signaling.

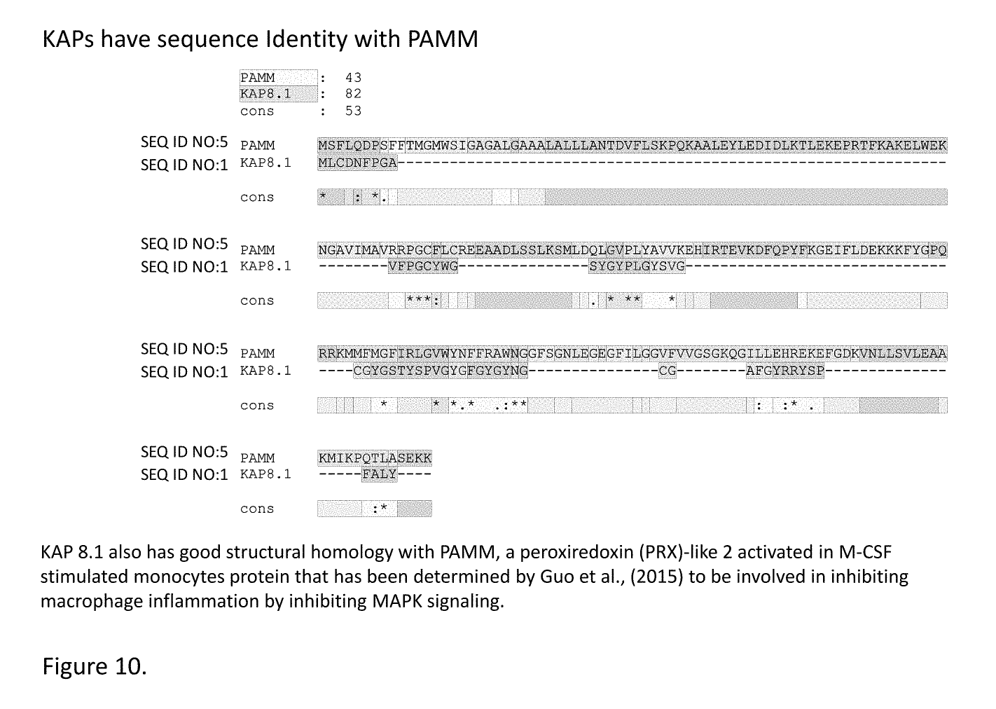

FIG. 11. ProgenaMatrix which contains KAPs and can induce the overexpression of anti-inflammatory cytokines IL-10 and IL-12.

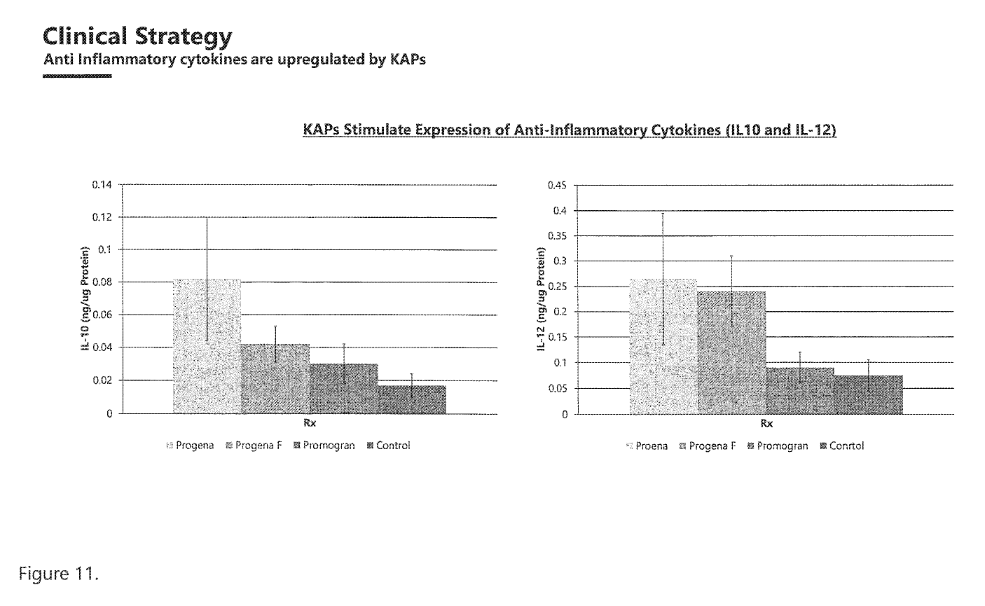

FIG. 12. Diabetic mouse model demonstrating that a ProgenaMatrix enriched in KAPs has increased levels of neutrophils (PMNs) and macrophage progenitor cells monocytes (both Ly6C^{hi} Inflammatory monocytes and Ly6C^{lo} anti-inflammatory monocytes) suggesting that KAPs prevent apoptosis and/or may promote more recruitment of macrophages.

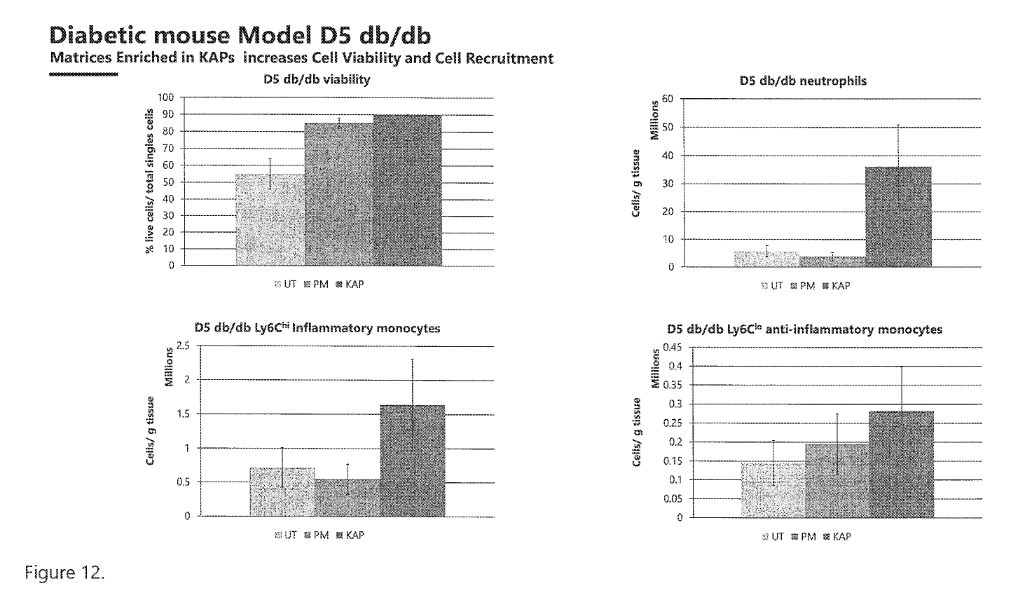

### DETAILED DESCRIPTION

Definition of Terms

The following terms as used herein shall have these defined meanings:

“BME” is 2-mercaptoethanol, also known as beta-mercaptoethanol.

“HPE” means human hair protein extract.

“HPEC” means human hair protein extract concentrate, which is HPE that has been concentrated by ultrafiltration.

“Hydrogel” means any non-liquid substance that is uniformly comprised of greater than about 40% water.

“Keratin” means any substance obtained from human hair or animal sources including wool, hair, hooves, fur, and feathers; no matter how crude, refined, processed, fractionated, purified, comminuted, chemically derivatized, blended, copolymerized, or otherwise changed or altered in any way by any means. It also encompasses mixtures of keratin with keratin associated proteins and mixtures of keratin with non-keratin substances that are not readily separable from keratin by practical means.

“Keratin wound dressing” means any product or experimental composition containing keratin that is intended for use in treating a wound to facilitate wound healing.

“KAP” or “KAPs” means keratin associated protein(s) and generally refers to keratin proteins that are not primarily contributory to the tensile strength of hair.

“KAP-enriched” or “KAP-enriched keratin proteins” means a keratin composition that comprises KAP that is in greater concentration than in the keratin substance within which it naturally exists or was originally obtained.

“Mesh” means any woven, knitted, braided, felted, or non-woven fabric made of any material by any means; any perforated or porous film of any porosity, pore size, composition or thickness; and any backing, substrate, or carrier membrane capable of being coated with the substances described in this specification.

“MWCO” means molecular weight cut off, which refers the specification of an ultrafiltration membrane that allows molecules having molecular weight below the specified molecular weight to pass through the membrane under pressure while retaining molecules that have a higher molecular weight than the specified molecular weight. For example, a 30 kDa MWCO membrane allows molecules lower than 30 kDa to pass through while retaining molecules that have molecular weight greater than 30 kDa.

“Periwound” means the margin of skin surrounding a wound that extends approximately one centimeter outward from the wound edge.

“ProgenaMatrix™” is a human keratin matrix manufactured from HPEC.

“Shindai” method or process means the conditions and methods utilized to produce HPE. Although undisclosed proprietary modifications to this process likely are being used by manufacturers of keratin wound dressings, the commonly accepted meaning of a Shindai extraction is the soaking of hair for 3 days at 50° C. in a pH 8.5 buffered solution of 5% BME containing 5 M urea and 2.5 M thiourea.

“Substrate” is the layer or layers of materials upon which a wound-contacting substance is coated or affixed to produce a wound dressing. There is no limitation to the nature or chemical composition of said materials. Examples of suitable materials can be found upon inspection of a plethora of commercially available wound dressing products that comprise synthetic polymers (for example: polyurethane, nylon, polyolefin, polyacrylate, polyvinyl chloride, polyvinyl alcohol, silicone rubber, styrene/butadiene rubber, and the like) and natural polymers (for example: collagen, keratin, silk, chitin, chitosan, cellulose, alginate, and the like).

“Wound” means skin that is missing at least its entire epidermal layer and at most is missing all its dermis and no more than about one-centimeter depth of subdermal flesh.

In one aspect, the invention is a hydrogel wound dressing equivalent to ProgenaMatrix™ in composition and physical properties except that it is KAP-enriched, thereby possessing enhanced efficacy relative to shortening the inflammatory phase of wound healing. The mechanism of action by which such enhanced efficacy operates resides in the previously discussed inhibitory action of certain KAPs toward excessive proteolytic enzyme activity observed in chronic wounds.

Several strategies for achieving KAP-enrichment have been described in the scientific literature or can readily be devised.¹ For example, it is well known that KAPs are generally of lower molecular weight than the bulk of hair keratins that give rise to the tensile strength and tenacity of human hair. These later keratins are in the molecular weight range of 40,000 to 65,000 Daltons (40-65 kDa), whereas most KAPs are below about 30,000 Daltons (30 kDa). The manufacturing process for making ProgenaMatrix™ begins with Shindai extraction of hair and subjecting the resultant HPE to an ultrafiltration step using a 30 kDa MWCO ultrafiltration membrane in a stirred pressure vessel to produce HPEC. The HPEC is then further processed to produce ProgenaMatrix™ and the HPEC-filtrate (passing through the ultrafiltration membrane) is discarded. About 90% of the proteins extracted in the Shindai process are retained in the HPEC and about 10% are discarded with the HPEC filtrate. It should be noted that all proteins passing through the 30 kDa MWCO membrane are likely of molecular weight lower than 30 kDa, but that not all proteins retained in the HPEC are necessarily above 30 kDa in molecular weight. Thus, it can be assumed that the HPEC solution contains KAPs and that the HPEC filtrate is KAP-enriched. ¹Santanu Deb-Choudhury, Jeffrey E. Plowman, Duane P. Harland, “Isolation and Analysis of Keratins and Keratin-Associated Proteins from Hair and Wool”, Methods in Enzymology, Volume 568 2016 Elsevier Inc. ISSN 0076-6879 All rights reserved. http://dx.doi.org/10.10.16/bs.mie.2015.07.018

Another strategy for obtaining KAP-enriched extracts of hair involves modifying the Shindai conditions. KAPs having a high glycine & tyrosine content (and hence a relatively low cysteine content) are of interest for the reasons discussed above. Since the cysteine amino acid residues in keratin proteins are solely responsible for the disulfide bridges that make hair keratins resistant to solvation, it is reasonable to expect that high glycine & tyrosine KAPs have a low disulfide crosslink density relative to high cysteine KAPs. This difference can be exploited to optimize extraction of low crosslink density KAPs from hair or from previously extracted keratin preparations by utilizing an extraction medium formulated to be less effective at breaking disulfide crosslinks.

Another useful approach to obtaining KAP-enriched keratin is simply to add ethanol or other alcohol to the Shindai extraction medium.² This reduces the yield of keratin proteins that otherwise would be extracted, but increases the KAP content of the recovered proteins. ²Toshihiro Fujii, Shunsuke Takayama, and Yumiko, Ito, “A novel purification procedure for keratin-associated proteins and keratin from human hair”, J. Biol. Macromol., 13(3), 92-106-2013

In a different aspect, the invention is a wound dressing in which the KAP-enriched keratin is coated on a mesh that serves as a supportive substrate. In this format the keratin is not a hydrogel. It is a thin layer that may or may not be a self-supporting membrane in the absence of the mesh substrate. The amount of KAP-enriched keratin coated on the mesh is preferably about 5 to 10 milligrams per square centimeter (5-10 mg/cm²), but could be from 1 to 20 mg/cm².

In certain aspects, the mesh is woven nylon fabric having about 70 μm pore openings and a weight of about 3 mg/cm², commercially available as 3 M Tegaderm Non-Adherent Contact Layer (3 M Company, 3 M Center, St. Paul, Minn. 55144).

Exemplary mesh openings can be from 5 μm to 2 mm or larger. In certain aspects, types of mesh that can be used include, but are not limited to, ADAPTIC TOUCH™ Non-Adhering Silicone Dressing; N-Terface® Wound Contact Material; Cardinal Health™ Petrolatum Emulsion Contact Layer; Cardinal Health™ Silicone Contact Layers; ColActive® Transfer (Wound Contact Layer); ComfiTel™ Silicone Contact Layer Dressing; CONFORMANT®2 Non-Adherent Contact Layer; COVRSITE® Wound Cover; Cuticell® Contact; Dermanet®; DRYNET* Wound Veil; Mepitel® One; Mepitel® Soft Silicone Wound Contact; Physiotulle® Wound Contact Layer; PROFORE® Wound Contact Layer Non-Adherent Dressing; Restore® Contact Layer FLEX; Silflex® Soft Silicone Wound Contact Layer; Silon-TSR®; Telfa™ Clear; and TRITEC™.

A process for making a KAP-enriched keratin hydrogel wound dressing includes: providing a KAP-enriched keratin in the form of a precipitate of fine particles; mixing said particles with HPEC and coating the mixture onto a silicone rubber surface; allowing the mixture to cure upon exposure to air into a stiff gel; and leaching out the HPEC chemicals in successive rinsing steps with alcohol and water. In this process, some or all the added KAP-enriched keratin may dissolve in the HPEC to which it is added, resulting in a finished product that may be clear or hazy, depending on its KAP-enriched keratin content. The ratio of KAP-enriched keratin to non-KAP-enriched keratin (comprising HPEC in this scenario) could from about 1:10 to about 10:1, respectively, or any fractional part thereof.

A process of forming a KAP-enriched keratin-coated mesh substrate wound dressing includes: mixing an aqueous solution of KAP-enriched keratin with a water-miscible organic solvent to obtain keratin particles; collecting the keratin particles; re-suspending the keratin particles in water to produce a keratin slurry; applying the keratin slurry to a substrate; and drying the keratin slurry-infused substrate to bond the keratin particles of the keratin slurry to the substrate.

The invention is further described by the following examples that are provided for illustration, not limitation.

### EXAMPLE 1

### KAP-enriched ProgenaMatrix™

HPEC filtrate is poured into a glass dish in a well-ventilated area and allowed to evaporate until reduced in volume by about one third, causing urea and thiourea to crystallize. The resultant concentrated clear liquid is separated from the crystals by filtration and poured into rapidly stirring denatured ethyl alcohol at the ratio of 10 parts by volume of alcohol for each one part by volume of HPEC-filtrate-concentrate. The resultant precipitate is collected by filtration and resuspended in a mixture of alcohol and water (1:1 by volume) and stirred to dissolve residual soluble substances. The solids are again collected by filtration and the process repeated until the weight of recovered rinse-residue upon evaporation of the filtrate is insignificant. The final washed precipitate is then dried by compression between layers of absorbent material, but not allowed to dry in air, which would otherwise cause the particles to fuse. The percent solids in the compressed precipitate is calculated by drying a small sample to constant weight. This value is used to calculate the desired ratio of KAP-enriched keratin to be added back to the HPEC from which it was originally removed. A mixture of KAP-enriched keratin (the compressed precipitate) and HPEC are prepared and the mixture poured into a shallow silicone rubber mold and allowed to cure in air, thereby gradually resulting in conversion of the liquid into a stiff, non-tacky gel. The time of curing is carefully monitored to guard against crystallization of urea, an undesirable phenomenon. Properly cured samples are immediately immersed in pure denatured alcohol and soaked for several hours, which removes most of the chemicals originally present in the HPEC. Subsequent rinsing is done in 1:1 alcohol:water and then in pure water until no further leaching of residual chemicals can be detected. The final, pure keratin hydrogel film is then packaged and sterilized in the same manner as ProgenaMatrix™ to provide a KAP-enriched product.

### EXAMPLE 2

### KAP-enriched Keratin-coated Mesh Wound Dressing

Clean hair clippings delipidized by Soxhlet extraction with a mixture of chloroform and methanol are soaked for 3 days at 50° C. in a pH 8.5 buffered solution of 5% BME containing 5 M urea and 2.5 M thiourea and also containing 10% by volume of pure ethyl alcohol. The resultant HPE obtained by removal of undissolved hair by filtration contained less protein than a corresponding HPE prepared without ethyl alcohol as an ingredient in the extraction medium but has a higher putative KAP content. This HPE is poured into a glass dish in a well-ventilated area and allowed to evaporate until reduced in volume by about one third, causing urea and thiourea to crystallize. The resultant concentrated clear liquid is separated from the crystals by filtration and poured into rapidly stirring denatured ethyl alcohol at the ratio of 10 parts by volume of alcohol for each one part by volume of HPEC-filtrate-concentrate. The resultant precipitate is collected by filtration and resuspended in a mixture of alcohol and water (1:1 by volume) and stirred to dissolve residual soluble substances. The solids are again collected by filtration and the process repeated until the weight of recovered rinse-residue upon evaporation of the filtrate is insignificant. The final washed precipitate is then dried by compression between layers of absorbent material, but not allowed to dry in air, which would otherwise cause the particles to fuse. The percent solids in the compressed precipitate is calculated by drying a small sample to constant weight. An amount of this compressed precipitate is resuspended in pure water with stirring and mixing to obtain a homogeneous slurry, with care to avoid the formation of foam. A piece of sheer nylon mesh is placed on a silicone rubber surface and the slurry poured over the mesh to create a uniformly thick pool of liquid, which is then allowed to evaporate to dryness. During the drying process the particles of KAP-enriched keratin settle into the mesh, causing them to be intimately infused therein and firmly bonded as the water evaporates. The side that was up during drying is now the wound-contact side of the dressing and the side that was down during drying is the mesh-side that forms a top layer upon application to a wound.

While the invention has been illustrated by a description of various aspects and while these aspects have been described in considerable detail, it is not the intention of the applicant to restrict or in any way limit the scope of the appended claims to such detail. Additional advantages and modifications will readily appear to those skilled in the art. Thus, the invention in its broader aspects is therefore not limited to the specific details, representative apparatus and method, and illustrative examples shown and described. Accordingly, departures may be made from such details without departing from the spirit or scope of applicants' general inventive concept.

What is claimed is:

1. A wound dressing comprising a KAP-enriched keratin material as the wound-contacting surface of the dressing.

2. A wound dressing of claim 1 wherein the KAP-enriched keratin material comprises a hydrogel composition.

3. A wound dressing of claim 1 where the KAP-enriched keratin material comprises a coating on a substrate.

4. A wound dressing of claim 3 wherein the substrate is a mesh.

5. A dressing of claim 1 wherein the KAPs comprise high glycine and tyrosine rich proteins.

6. A dressing of claim 5 wherein the KAPs comprise one or more keratin associated proteins known by the number designations of 7.1, 8.1, 19.1, and 19.2.

7. A process of forming a KAP-enriched keratin wound dressing, the process comprising:
separating KAP-enriched keratin from a keratin material;
mixing an aqueous solution of KAP-enriched keratin with a water-miscible organic solvent to obtain keratin particles;
purifying said particles and depositing them on a substrate;
drying the substrate to bond the KAP-enriched keratin particles to the substrate.

8. A process of forming a KAP-enriched hydrogel wound dressing, the process comprising:
separating KAP-enriched keratin from a keratin material;
mixing an aqueous solution of KAP-enriched keratin with a water-miscible organic solvent to obtain keratin particles;
purifying said particles and removing liquid;
mixing the purified particles with HPEC;
curing the HPEC into a hydrogel and purifying by rinsing with alcohol and water.
